(19) United States
(12) Patent Application Publication
(10) Pub. No.: US 2023/0255894 A1
(43) Pub. Date: Aug. 17, 2023

(54) METHOD OF PREPARING LIPID VESICLES

(71) Applicant: MICROPORE TECHNOLOGIES LIMITED, Cleveland (GB)

(72) Inventors: Alex Kerr, Cleveland (GB); Sam Trotter, Cleveland (GB); David Hayward, Cleveland (GB)

(73) Assignee: MICROPORE TECHNOLOGIES LIMITED, Cleveland (GB)

(21) Appl. No.: 18/016,143

(22) PCT Filed: Jul. 21, 2021
(86) PCT No.: PCT/GB2021/051887

§ 371 Date: Jan. 13, 2023

(30) Foreign Application Priority Data
Jul. 22, 2020 (GB) 2011367.6

(51) Int. Cl. A61K 9/51; A61K 9/127; A61K 31/661; B01D 69/04

(52) U.S. Cl. CPC A61K 9/5192; A61K 9/1277; A61K 9/1271; A61K 31/661; B01D 69/04; B01D 2325/02

(57) ABSTRACT

There is described a method of preparing lipid vesicles, said method comprising dispersing a first liquid phase in a second liquid phase; wherein said first liquid phase comprises a lipid phase and said second liquid phase comprises an aqueous phase; or said first liquid phase comprises an aqueous phase and said second liquid phase comprises a lipid phase; said method comprising controlling provision of the first liquid phase in a first flow direction to a membrane, said membrane defining a plurality of pores; and controlling provision of the second liquid phase to the membrane in a crossflow to the first flow direction, via the plurality of pores, to form a lipid vesicle suspension.

### FIELD OF THE INVENTION

The present invention relates to a novel method of preparing lipid vesicles.

More particularly, the present invention relates to a method of preparing lipid vesicles for use in the field of therapy, in particular, the delivery of bioactive agents, such as a therapeutic agent (drug), mRNA (vaccine) and the like.

### BACKGROUND TO THE INVENTION

Liposomes and lipid nanoparticles (LNPs) are similar by design, but slightly different in composition and function. Both are lipid nanoformulations and excellent drug delivery vehicles, transporting a payload within a protective, outer layer of lipids.

Traditional liposomes include one or more rings of lipid bilayer surrounding an aqueous core or pocket.

LNPs are liposome-like structures, but not all LNPs have a contiguous bilayer that would qualify them as lipid vesicles or liposomes. Some LNPs assume a micelle-like structure, encapsulating payload molecules in a non-aqueous core.

LNPs are especially geared towards encapsulating a broad variety of nucleic acids (RNA and DNA); and as such, they are the most popular non-viral gene delivery system, for example, used in vaccine delivery.

Generally, vaccines use low doses of a specific antigen or an antigenic agent to build up resistance in a host, such that the host is able to combat the effects of larger doses of the antigen or similar antigenic agents.

Antigens used in vaccines are usually parts of whole organisms or denatured toxins (toxoids) that induce the production of antibodies. However, only some of the antibodies produced bind to the target organism or toxin, since, in most cases, the antigen used in the vaccine differs structurally from the target.

Conventional vaccines use attenuated and inactivated pathogens. However, more recently, messenger RNA (mRNA) vaccines have been developed as an alternative to conventional vaccines. The use of mRNA has several beneficial features over conventional vaccines. Since mRNA is a non-infectious platform, there is no potential risk of infection; and mRNA is degraded by normal cellular processes. In addition, mRNA vaccines have the potential for rapid, inexpensive and scalable manufacturing. Vaccines generally comprise therapeutic nucleic acids, e.g., messenger RNA (mRNA), antisense oligonucleotides, ribozymes, DNAzymes, plasmids, immune stimulating nucleic acids, antagomir, antimir, mimic, supermir and aptamers. However, the delivery of nucleic acids to affect a desired response in a biological system presents many challenges. Nucleic acid based therapeutics have enormous potential but there remains a need for more effective delivery of nucleic acids to appropriate sites within a cell or organism in order to realize this potential. The limited bioavailability of antigens poses limitations to vaccine development.

There has been increased interest in the delivery of antigenic agents, particularly in the search for a SARS-CoV-2 (COVID-19) vaccine. McKay et al reports the investigation of a vaccine comprising a self-amplifying RNA encoding the SARS-CoV-2 spike protein, encapsulated within a proprietary lipid nanoparticle (LNP) composition. The LNPs had a mean hydrodynamic diameter of ˜75 nm with a polydispersity index of <0.1. McKay identifies that there is a need for this development to be rapidly scalable.

The key advantages of LNPs as a vaccine delivery system are their ability to protect genetic material encoding antigens against degradation, to control the release of the genetic material, enhance cellular uptake, and improve antigen-specific immune responses.

Lipid vesicles are of interest in the pharmaceutical industry for delivery of therapeutic agents, such as, anti-cancer drugs, including RNA delivery systems; antibiotics, gene therapies, anaesthetics and anti-inflammatory drugs. Physicochemical properties of lipid vesicles such as size, charge, and membrane fluidity can be modified to improve their ability to successfully deliver their payload.

Liposomes as parenteral drug delivery carriers are currently being utilized in the pharmaceutical industry. Liposomes have proven to be useful in delivering therapeutic agents for the treatment of (amongst other conditions) cancer, macular degeneration and fungal infections. To date, there are various types of liposomes that are adapted for these different applications, that include the delivery of a variety of types of therapeutic agent, including gene-delivery, siRNA-delivery, protein/peptide delivery and small molecule delivery. Depending on the application and target, there will be differences in the liposome formulations such as in lipid type/composition, affecting the physicochemical properties of the liposomes such as size, charge, and membrane fluidity, which can be modified to improve their ability to reach the desired target and deliver their payload.

Liposomes are generally formed when amphiphilic lipids organize themselves spontaneously in bilayer vesicles as a result of interactions between phospholipids and water. As these lipid vesicles possess lipophilic and hydrophilic portions, they can entrap substances with different polarities either in the phospholipid bilayer (hydrophobic substances) or the aqueous compartment (hydrophilic substances) or at the bilayer interfaces, which can modify physicochemical properties of the phospholipids and can enhance biological activity of entrapped compounds.

The properties of liposomes such as the hydrodynamic radius (size), zeta-potential, lipid-packing, encapsulation efficiency, and external modifications (such as polymer coatings) are important in formulating an efficacious drug delivery system. When considering in vivo applications of liposomes, the correct size of liposomes is one property that is vital in order to deliver the liposomes to different locations in the body. For example, liposomes with an approximate diameter of <100 nm are known to accumulate at cancer sites as a result of the enhanced permeability retention (EPR) effect, whereas very small liposomes or larger liposomes are filtered or taken up elsewhere in the body, respectively.

Liposomes can be classified via their size and lamellarity:
Multilamellar vesicles (MLVs)—1-5 μm
Large unilamellar vesicles (LUVs)—100-250 nm
Small unilamellar vesicles (SUVs)—20-100 nm

Generally, multilamellar vesicles (MLVs) are relatively unpredictable and/or have uncontrolled morphology, and are not effective hydrophilic drug carriers due to the small core volume. The most desirable liposome for drug delivery, e.g. vaccines and the like, are LUVs and SUVs.

Liposome properties are highly dependent on the processing conditions of the formulation, and any alterations in these processing conditions will lead to differences in the final formulation. Therefore, it is important to develop a manufacturing system that can accurately and predictively produce liposomes based on the user's requirements and which can be scaled up. As identified by McKay above, there is a need for the production of antigen carrying liposomes to be rapidly scalable.

Several techniques have been reported in the literature for the preparation of liposomes. Ethanol injection is one of the techniques most frequently used to produce liposomes. In the ethanol injection technique, an ethanolic solution of lipids is rapidly injected into an aqueous medium, usually a buffered system, through a needle, dispersing the phospholipids throughout the medium. This immediate dilution of ethanol in the aqueous phase causes the lipid molecules to precipitate and form bilayer planar fragments which further transform into a liposomal system. This is a mild procedure which affords a reasonably homogeneous vesicle population, although rather diluted. The ethanol injection method was first reported in the early 1970s by Batzri and Korn (Batzri S, et al “Single bilayer liposomes prepared without sonication” Biochim Biophys. Acta, 1973; 298(4); 1015-1019).

US Patent application No. 2004/0032037 (Polymun) describes a method for producing lipid vesicles using the ethanol injection method. In the method described in US '037 the polar (aqueous) phase is pumped from a storage container into a pipe system connected thereto and comprising one or more pipes. Each pipe through which the polar phase flows and which leads away from the storage container contains, at a predetermined point, at least one, laterally arranged hole or orifice, which is connected through the pipe wall and on the outside to at least one feed pipe for the pressure-controlled feeding of the lipid phase dissolved in a suitable solvent. The method described therein creates unilamellar vesicles having a narrow size distribution and without the action of mechanical agitating or dispersing aids.

European Patent application No. 3711749 (Polymun) describes a method of producing lipid nanoparticles having an average diameter of less than 100 nm, comprising pumping an aqueous buffer solution through a 1^{st} tube by a 1^{st} HPLC pump, pumping an organic lipid solution through a 2^{nd} tube by a 2^{nd} HPLC pump, wherein the 2^{nd} tube intersects the 1^{st} tube perpendicularly within a mixing module, and wherein the organic lipid solution is mixed with the aqueous solution in a turbulent flow within the mixing module.

Chinese patent application No. CN103637993 describes the preparation of monodisperse nanometer cefquinoxime sulfate liposomes using membrane emulsification techniques.

International Patent application No. WO 2019/092461 describes a crossflow apparatus for producing a suspension or dispersion by dispersing a first phase through a membrane in a second phase.

### SUMMARY OF THE INVENTION

Therefore, there is a need for a method and apparatus for the particularly mild production of lipid vesicles, e.g. liposomes and LNPs, which can be scaled up, and which can optionally be continuous process. The method should provide homogeneously distributed liposome vesicle preparations in a reproducible manner.

Thus, the present invention allows the scaled up and/or continuous production of lipid vesicles utilising established methods, such as ethanol injection.

Furthermore, it has been surprisingly found that a crossflow membrane emulsification apparatus (AXF), utilising a tubular membrane, can suitably be used for the production of lipid vesicles.

According to a first aspect of the invention there is provided a method of preparing lipid vesicles, said method comprising dispersing a first liquid phase in a second liquid phase;
wherein said first liquid phase comprises a lipid phase and said second liquid phase comprises an aqueous phase; or said first liquid phase comprises an aqueous phase and said second liquid phase comprises a lipid phase;
said method comprising controlling provision of the first liquid phase in a first flow direction to a membrane, said membrane defining a plurality of pores; and controlling provision of the second liquid phase to the membrane in a crossflow to the first flow direction, via the plurality of pores, to form a lipid vesicle suspension.

In one aspect of the invention the first liquid phase comprises a lipid phase and the second liquid phase comprises an aqueous phase.

In another aspect of the invention the first liquid phase comprises an aqueous phase and the second liquid phase comprises a lipid phase.

The lipid vesicles produced by the method of the invention may be liposomes or lipid nanoparticles (LNPs). According to one aspect of the invention the lipid vesicles are liposomes. According to another aspect of the invention the lipid vesicles are LNPs.

According to a further aspect of the invention there is provided a method of preparing lipid vesicles, said method comprising dispersing a first liquid phase in a second liquid phase, wherein said first liquid phase comprises a lipid phase;
wherein said method uses a crossflow emulsification apparatus (AXF); said crossflow emulsification apparatus comprising:
an outer tubular sleeve provided with a first inlet at a first end; a lipid vesicle outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate; and
controlling provision of the first liquid phase to the tubular membrane; and controlling provision of a second liquid phase to the tubular membrane via the plurality of pores to form a lipid vesicle suspension.

In one aspect of the invention the first liquid phase comprises a lipid phase and the second liquid phase comprises an aqueous phase.

In another aspect of the invention the first liquid phase comprises an aqueous phase and the second liquid phase comprises a lipid phase.

According to this method of the invention the lipid vesicles produced by the method of the invention may be liposomes or lipid nanoparticles (LNPs). According to one aspect of the invention the lipid vesicles are liposomes. According to another aspect of the invention the lipid vesicles are LNPs.

According to a yet further aspect of the invention the aqueous phase may include one or more active agents. In this aspect of the invention the product of the method of preparing lipid vesicles is a lipid vesicle composition comprising of a lipid bilayer encapsulating an aqueous core. The aqueous core may include one or more active agents or the lipid vesicles may be produced unloaded and loaded afterwards (active loading). Loading of active agents can be attained either by passive loading i.e. the active agent is encapsulated during formation of the lipid vesicle; or active loading, i.e. the active agent is loaded after formation of the lipid vesicle.

Thus, according to one aspect of the invention the lipid vesicles are produced loaded (passive loading).

According to another aspect of the invention the lipid vesicles are produced unloaded and loaded afterwards (active loading).

For example, hydrophilic active agents are distributed homogenously in an aqueous phase, both inside and outside the lipid vesicle; whereas hydrophobic active agents can be directly combined into lipid vesicles during vesicle formation, and the amount of uptake and retention is governed by active agent/lipid interactions.

In active loading, lipid vesicles are generated containing a transmembrane gradient, i.e. the phase inside the lipid vesicle and outside are different, so that subsequently the active agent dissolved in the exterior phase can permeate across the lipid vesicle wall. The transmembrane gradient can be a pH gradient, a concentration gradient, an ion gradient, and the like. Active loading using an ion gradient, will often comprise a sulfate ion gradient, e.g. by utilising ammonium sulfate. An ion gradient can be achieved by replacing the first buffer, in which the lipid vesicles are formed, by a second buffer, e.g. through dialysis or ultrafiltration. Addition of the second buffer, and then concentrating the suspension, provides the gradient, as the first buffer will still be inside the formed lipid vesicles.

The choice of loading, i.e. active or passive loading, may influence the choice of aqueous phase buffer.

A pH gradient can be achieved by, for example, running a system in an acidic buffer (pH 4), e.g. citrate, and replacing the buffer with a suitable aqueous phase buffer of a different, e.g. higher, pH. Examples of suitable aqueous phase buffers include, but shall not be limited to, MES (2-(No-morpholino)ethianesulfonic acid), citrate, phosphate, acetate, HEPES (4-(2-hydroxyethyl)-1-piperazineethanesulfonic acid), TRIS (tris(hydroxymethyl)aminomethane) and PBS (phosphate-buffered saline); and combinations thereof.

The active agent may be encapsulated in the lipid portion of the lipid vesicle or an aqueous space enveloped by some or all of the lipid portion of the lipid vesicle, thereby protecting it, for example, from enzymatic degradation.

According to this aspect of the invention the lipid vesicles produce by the method of the invention may be liposomes or lipid nanoparticles (LNPs). According to one aspect of the invention the lipid vesicles are liposomes. According to another aspect of the invention the lipid vesicles are LNPs. When the lipid vesicles are liposomes then the solvent phase may comprise an aqueous phase. When the lipid vesicles comprise LNPs the solvent phase may comprise a non-aqueous solvent phase.

When the one or more active agents is hydrophilic, then the solvent phase may comprise an aqueous phase. When the one or more active agents is hydrophobic, then the hydrophobic active agent will be in the lipid phase, and the lipid is dissolved in a hydrophobic solvent, e.g. an organic solvent. The aqueous phase inside the lipid vesicle will have essentially no active agent in it.

It will be understood by the person skilled in the art that any conventionally known soluble active agents may be encapsulated according to the method of the present invention. However, in a particular embodiment of the invention the one or more active agents is a bioactive agents, such as a therapeutic agent (drug), vaccine and the like. In one aspect of the invention the bioactive agent may be a therapeutic nucleic acid, such as one encoding an antigen. Therapeutic nucleic acids include, e.g., messenger RNA (mRNA), antisense oligonucleotides, ribozymes, DNAzymes, plasmids, immune stimulating nucleic acids, antagomir, antimir, mimic, supermir and aptamers. Suitable antigens are any chemicals that are capable of producing an immune response in a host organism. The antigen may be a suitable native, non-native, recombinant or denatured protein or peptide, or a fragment thereof, that is capable of producing the desired immune response in a host organism. Host organisms are preferably animals (including mammals), more preferably humans. The antigen can be of a viral, bacterial, protozoal or mammalian origin. Antigens are generally known to be any chemicals (typically proteins or other peptides) that are capable of eliciting an immune response in a host organism. More particularly, when an antigen is introduced into a host organism, it binds to an antibody on B cells causing the host to produce more of the antibody. For a general discussion of antigens and the immune response, see Kuby, J., Immunology 3^{rd} Ed. W.H. Freeman & C. NY (1997).

Lipid vesicles of the invention, e.g. liposome or LNPs, can be formed from a single lipid or from a mixture of lipids.

It will be understood by the person skilled in the art that more conventional pharmaceuticals may be delivered using neutral lipid vesicles along with positively or negatively charged lipids; and any combination thereof. Examples of such neutral structural lipids include, but shall not be limited to, sphingosylphosphorylcholine (SPC), L-α-hydrogenated phosphatidylcholine (HSPC), distearoylphosphatidylcholine (DSPC) and 1-palmitoyl-2-oleoylphosphatidylcholine, (POPC), and the like; and combinations thereof.

Lipid vesicles and liposomal particles are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter 100≤nm; and LUVs have a diameter>100 nm.

Lipid vesicles of the present invention may preferably be SUVs or LUVs with a diameter in the range of 50-220 nm. For a composition comprising a population of SUVs or LUVs with different diameters: (i) at least 80% by number should have diameters in the range of 20-220 nm; (ii) the average diameter of the population is ideally in the range of 40-200 nm, and/or (iii) the diameters should have a polydispersity index (PDI)≤0.3, e.g. from about 0.02 to about 0.3, preferably between 0.02 and 0.2. The lipid vesicle may be substantially spherical.

Various amphiphilic lipids can form bilayers in an aqueous environment to encapsulate, for example, a lipid vesicle with a solvent core containing one or more therapeutic nucleic acids. These lipids can have an anionic, cationic, zwitterionic or ionisable (variable charge) hydrophilic head group. Lipid vesicles prepared by the method of the invention for the delivery of nucleic acids may comprise a lipid having a pKa in the range of 5.0 to 7.6. Some lipids with a pKa in this range may include a tertiary amine. For example, they may comprise 1,2-dilinoleyloxy-N,N-dimethyl-3-aminopropane or 1,2-dilinolenyloxy-N,N-dimethyl-3-aminopropane. Another suitable lipid having a tertiary amine is 1,2-dioleyloxy-N,Ndimethyl-3-aminopropane. For the delivery of nucleic acids cationic lipids, such as, DDA (dimethyl dioctadecyl ammonium bromide) or DOTAP (1,2-dioleoyl-3-trimethylammonium-propane) may suitably be used.

Particularly useful lipid vesicles use phospholipids which may optionally include unesterified cholesterol in the lipid vesicles formulation. Unesterified cholesterol may be used to stabilise the lipid vesicles and any other compound that stabilises lipid vesicles may replace the cholesterol. Other lipid vesicles stabilising compounds are known to those skilled in the art. The use of stabilised lipid vesicles may result in limiting the electrostatic association between the nucleic acid and the lipid vesicles. Consequently, most of the nucleic acid may be sequestered in the interior of the lipid vesicles.

Phospholipids that are preferably used in the preparation of lipid vesicles are those with at least one head group selected from the group consisting of phosphoglycerol, phosphoethanolamine, phosphoserine, phosphocholine and phosphoinositol.

In one aspect of the invention the lipid vesicle may comprise both a lipid portion and a polymer portion, e.g. a pegylated lipid vesicle. A “pegylated lipid” specifically refers to a lipid or lipid vesicle comprising both a lipid portion and a polyethylene glycol portion. It will be understood by the person skilled in the art that lipid vesicles comprising a lipid and a polymer portion other than polyethylene glycol are within the scope of the present invention. Pegylated lipids include, but shall not be limited to, 1-(monomethoxy-polyethyleneglycol)-2,3-dimyristoylglycerol (PEG-DMG), pegylated diacylglycerol (PEG-DAG), e.g. 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG), e.g. 4-O-(2′,3′-di(tetradecanoyloxy)propyl-1-O-(\acute{ω}-methoxy(polyethoxy)ethyl)butanedioate (PEG-S-DMG), a pegylated ceramide (PEG-cer), PEG dialkoxypropylcarbamate, e.g. \acute{ω}-methoxy(polyethoxy)ethyl-N-(2,3-di(tetradecanoxy)propyl)carbamate or 2,3-di(tetradecanoxy)propyl-N-(\acute{ω}-methoxy(polyethoxy)ethyl)carbamate or distearoyl-sn-glycero-3-phosphoethanolamine-N-[amino(polyethylene glycol) 2000]; and combinations thereof.

The amount of lipid used to form the lipid vesicles depends on the active agent being used but is typically in a range from about 0.01 g to about 0.5 g per dose of e.g. vaccine. The amount of lipid used may be about 0.1 g per dose. When unesterified cholesterol is also used in the lipid vesicle formulation, the preferred amount of cholesterol or a stabilising compound other than cholesterol can readily be determined by the person skilled in the art.

Techniques for preparing suitable lipid vesicles are well known in the art. One such method involves mixing an ethanolic solution of the lipids with an aqueous solution of the active agent. It will be understood by the person skilled in the art that since the technique relies on solvent-water miscibility, other water miscible solvents may suitably be used, for example, C₁-C₆ alkanols, such as, methanol, ethanol, propanol, butanol, pentanol, hexanol, and the like.

The method of the present invention is adaptable to large-scale, commercial production of formulations of nanoscale lipid vesicles, particularly of those that comprise substantially homogenous lipid vesicle particle sizes that may be no bigger than about 220 nm in diameter. For example, more than 90% (volume weighted, e.g. as determined by dynamic light scattering) of lipid vesicles are less than about 220 nm; or more than 99% less than about 220 nm. Such sized particles can be readily filter sterilised according to industry-approved clinical manufacturing standards.

A preparation of such homogenously-sized lipid vesicles can be made according to the present invention by controlling the concentration of organic solvent, keeping it essentially constant at, and following, the formation of the lipid vesicles. By controlling solvent concentration it is possible to control the size of lipid vesicle particles that are formed when the lipid solution and solvent (aqueous or non-aqueous solvent suitable for use in lipid vesicle formation). By controlling solvent concentration during mixing/lipid phase addition, the lipid vesicle size can be controlled. In a continuous process, this may be referred to as the flow-rate-ratio (FRR). The FRR is an important process attribute. At high solvent concentration, the lipid vesicles may be malleable/changeable, therefore dilution may be used to reduce solvent concentration in order to set the lipid vesicle size. This may be combined with using dilution to change the buffer system for active loading of RNA species.

In general, reducing the polarity of the solvent will increase the size of the lipid vesicle, i.e. by lowering the FRR. For example, a process for the preparation of lipid vesicles may be run at a ratio of aqueous:organic of about 1:1. Thus, more organic solvent, e.g. ethanol and lower overall polarity of the mixed solvent:water system produced, creates larger lipid vesicles. Furthermore, an increase in the polarity of the solvent causes the lipids to become progressively less soluble and self-assemble into planar lipid bilayers.

In the method of the present invention the crossflow membrane emulsification uses the flow of a continuous phase, to sweep and evenly mix flows of a disperse phase coming through the membrane pores. This contrasts with known prior art systems which use turbulent flow for lipid vesicle production. The mixing or micromixing comprises a controlled mixing of phases.

The position of the lipid vesicle outlet may vary depending upon the direction of flow of the disperse phase, i.e. from inside the membrane to outside or from outside the membrane to inside. If the flow of the disperse phase is from outside the membrane to inside then the lipid vesicle outlet will generally be at a second end of the tubular sleeve. If the flow of the disperse phase is from inside the membrane to outside then the lipid vesicle outlet may be a side branch or at the end.

In one aspect of the invention the crossflow apparatus includes an insert as herein described and the first inlet is a continuous phase first inlet and the second inlet is a disperse phase inlet; such that the disperse phase travels from outside the tubular membrane to inside.

In another aspect of the invention the crossflow apparatus does not include an insert and the first inlet is a disperse phase first inlet and the second inlet is a continuous phase inlet; such that the disperse phase travels from inside the tubular membrane to outside.

In one aspect of the invention the disperse phase is the lipid phase and the continuous phase is a solvent phase. The solvent phase may optionally include one or more active agents as herein defined.

In another aspect of the invention the disperse phase is the solvent phase and the continuous phase is a lipid phase. The solvent phase may optionally include one or more active agents as herein defined.

When an insert is present and the tubular membrane is positioned inside the outer sleeve, the spacing between the insert and the tubular membrane may be varied, depending upon the laminar conditions desired, etc. Generally, the insert will be located centrally within the tubular membrane, such that the spacing between the insert and the membrane will comprise an annulus, of equal or substantially equal dimensions at any point around the insert. Thus, for example, the spacing may be from about 0.05 to about 10 mm (distance between the outer wall of the insert and the inner wall of the membrane), from about 0.1 to about 10 mm, from about 0.25 to about 10 mm, or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

When the tubular membrane is positioned inside the outer sleeve, the spacing between the tubular membrane and the outer sleeve may be varied. Generally, the tubular membrane will be located centrally within the outer sleeve, such that the spacing between the membrane and the sleeve will comprise an annulus, of equal or substantially equal dimensions at any point around the tubular membrane. Thus, for example, the spacing may be from about 0.5 to about 10 mm (distance between the outer wall of the membrane and the inner wall of the sleeve), or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

In an alternative embodiment of the invention the insert is tapered, such that the spacing between the insert and the tubular membrane may be divergent along the length of the membrane. The spacing and the amount of divergence varied, depending upon the gradient of the tapered insert, the laminar conditions/flow velocities desired, size distribution, etc. It will be understood by the person skilled in the art that depending upon the direction of taper, the spacing between the insert and the tubular membrane may be divergent or convergent along the length of the membrane. The use of a tapered insert may be advantageous in that a suitable taper may allow the laminar flow to be held constant for a particular formulation and set of flow conditions. Thus, the tapered insert may be used to control variation in mixing conditions resulting from changes in fluid properties, such as viscosity, as the ethanol or other solvent and lipid concentration increases through its path along the length of the membrane.

In an alternative embodiment of the invention the crossflow apparatus may comprise more than one tubular membrane located inside the outer tubular sleeve, i.e. a plurality of tubular membranes. When a plurality of tubular membranes is provided, each membrane may optionally have an insert, as herein described, located inside it. A plurality of membranes may be grouped as a cluster of membranes positioned alongside each other. Desirably the membranes are not in direct contact with each other. It will be understood that the number of membranes may vary depending upon, inter alia, the nature of the materials to be produced. Thus, by way of example only, when a plurality of tubular membranes is present, the number of membranes may be from 2 to 100.

The inclined second inlet provided in the outer tubular sleeve will generally comprise a branch of the tubular sleeve and may be perpendicular to the longitudinal axis of the tubular sleeve. The position of the branch or second inlet may be varied and may depend upon the plane of the membrane. In one embodiment the position of the branch or second inlet will be substantially equidistant from the inlet and the outlet, although it will be understood by the person skilled in the art that the location of this second inlet may be varied. It is also within the scope of the present invention for more than one branch inlet to be provided. For example the use of a dual branch may suitably allow for bleeding the continuous phase during priming, or flushing for cleaning, or drainage/venting for sterilisation.

The inlet and outlet ends of the outer sleeve will generally be provided with a seal assembly. Although the seal assemblies at the inlet and outlet ends of the outer sleeve may be the same or different, preferably each of the seal assemblies is the same. Normal O-ring seals involve the O-ring being compressed between the two faces on which the seal is required—in a variety of geometries. Commercially available O-ring seals are provided with different groove options with standard dimensions. Each seal assembly will comprise a tubular ferrule provided with a flange at each end. A first flange, located at the end adjacent to the outer sleeve (when coupled) may be provided with a circumferential internal recess which acts as a seat for an O-ring seal. When the O-ring seal is in place, the O-ring seal is adapted to be located around the end of the insert (when present) and within a recess in the outer sleeve to seal against leakage of fluid from within any of the elements of the crossflow apparatus. However, the O-ring seal used in the present invention is designed to allow a loose fit as the membrane slides through the O-rings. This arrangement is advantageous in that it avoids two potential problems while installing the membrane tube:
(1) the potential for crushing the thin membrane tube during installation; and
(2) the potential for the thin membrane tube to cut off the curved surface of the O-ring.

With the O-ring seal used in the present invention, when the end ferrules are clamped onto the outer sleeve they squeeze the sides of the O-rings causing them to deform and press onto the outer surface of the tubular membrane and the inner surface of the sleeve, to form a seal. This requires careful dimensioning and tolerances.

However, it will be understood by the person skilled in the art that other means of making seal may suitably be used, for example, use of a screwed fitting tightened to a particular torque which would avoid the need for close tolerances; or clamping parts to a particular force followed by welding (which may be particularly suitable when using a plastic crossflow apparatus).

The internal diameter of the tubular membrane may be varied. In particular, the internal diameter of the tubular membrane may vary depending upon whether or not an insert is present. Generally, the internal diameter of the tubular membrane will be fairly small. In the absence of an insert the internal diameter of the tubular membrane may be from about 1 mm to about 10 mm, or from about 2 mm to about 8 mm, or from about 4 mm to about 6 mm. When the tubular membrane is intended for use with an insert, the internal diameter of the tubular membrane may be from about 5 mm to about 50 mm, or from about 10 mm to about 50 mm, or from about 20 mm to about 40 mm, or from about 25 mm to about 35 mm. Higher internal diameter of the tubular membrane may only be capable of being subjected to lower injection pressure. The upper limit of the internal diameter of the tubular membrane may depend upon, inter alia, the thickness of the membrane tube, since the cylinder needs to be able to cope with the external injection pressure, and whether it's possible to drill consistent holes through that thickness. The chamber inside the cylindrical membrane usually contains the continuous phase liquid.

In contrast to membrane emulsification using oscillating membranes, in the present invention the membrane, the sleeve and the insert are generally stationary.

As described herein in prior art membranes, such as those described in WO2012/094595 comprise pores in the membrane that are conical or concave in shape. One example is that the pores in the membrane can be laser drilled. Laser drilled membrane pores or through holes will be substantially more uniform in pore diameter, pore shape and pore depth. The profile of the pores may be important, for example, a sharp, well defined edge around the exit of the pore is preferable. It may be desirable to avoid a convoluted path (such as results from sintered membranes) in order to minimise blockage, reduce feed pressures (cf. mechanical strength), and keep an even flowrate from each pore. However, as discussed herein, it is within the scope of the present invention to use pores in which the internal bore is non-circular (for example rectangular slots) or convoluted (for example tapered or stepped diameter to minimise pressure drop).

In the membrane the pores may be uniformly spaced or may have a variable pitch. Alternatively, the membrane pores may have a uniform pitch within a row or circumference, but a different pitch in another direction.

The pores in the membrane may vary. By way of example only, the pores in the membrane may have a pore diameter of from about 1 μm to about 200 μm, or from about 1 μm to about 100 μm, or about 10 μm to about 100 μm, or about 20 μm to about 100 μm, or about 30 μm to about 100 μm, or about 40 μm to about 100 μm, or about 50 μm to about 100 μm, or about 60 μm to about 100 μm, or about 70 μm to about 100 μm, or about 80 μm to about 100 μm, or about 90 μm to about 100 μm. In a further embodiment of the invention the pores in the membrane may have a pore diameter of from about 1 μm to about 40 μm, e.g. about 3 μm, or from about 5 μm to about 20 μm, or from about 5 μm to about 15 μm.

In the membrane the shape of the pores may be substantially tubular. However, it is within the scope of the present invention to provide a membrane with uniformly tapered pores. Such uniformly tapered pores may be advantageous in that their use may reduce the pressure drop across the membrane and potentially increase throughput/flux. It is also within the scope of the present invention to provide a membrane in which the diameter is essentially constant, but the internal bore is non-circular (for example rectangular slots) or convoluted (for example tapered or stepped diameter to minimise pressure drop), providing pores with a high aspect ratio.

The interpore distance or pitch may vary depending upon, inter alia, the pore size; and may be from about 1 μm to about 5,000 μm, or from about 1 μm to about 1,000 μm, or from about 2 μm to about 800 μm, or from about 5 μm to about 600 μm, or from about 10 μm to about 500 μm, or from about 20 μm to about 400 μm, or from about 30 μm to about 300 μm, or from about 40 μm to about 200 μm, or from about 50 μm to about 100 μm, e.g. about 75 μm.

The surface porosity of the membrane may depend upon the pore size and may be from about 0.001% to about 20% of the surface area of the membrane; or from about 0.01% to about 20%, or from about 0.1% to about 20%, or from about 1% to about 20%, or from about 2% to about 20%, or from about 3% to about 20%, or from about 4% to about 20%, or from about 5% to about 20, or from about 5% to about 10%.

The arrangement of the pores may vary depending upon, inter alia, pore size, throughput, etc. Generally, the pores may be in a patterned arrangement, which may be a square, triangular, linear, circular, rectangular or other arrangement. In one embodiment the pores are in a square arrangement.

It will be understood that the apparatus of the invention; and in particular the membrane, may comprise known materials, such as glass; ceramic; metal, e.g. stainless steel or nickel; polymer/plastic, such as a fluoropolymer; or silicon. The use of metals, such as stainless steel or nickel, or polymer/plastic, such as a fluoropolymer is advantageous in that, inter alia, the apparatus and/or membranes may be subjected to sterilisation, using conventional sterilisation techniques known in the art, including gamma irradiation where appropriate. The use of polymer/plastic material, such as a fluoropolymer, is advantageous in that, inter alia, the apparatus and/or membrane may be manufactured using injection moulding techniques known in the art.

As described herein an insert may be included in the membrane to facilitate even flow distribution. However, it is within the scope of the crossflow apparatus of the present invention for the insert to be absent. When an insert is present, the furcation plate may be adapted to split the flow of continuous phase or the disperse phase into a number of branches. Whether the furcation plate splits the continuous phase or the disperse phase will depend upon the direction of flow of the continuous phase, i.e. whether the continuous phase flows through the first inlet or the second inlet.

Although the number of furcation plates may be varied, the number selected should be suitable lead to even flow distribution and (at the lipid vesicle outlet end) not have excessive shear. Preferably, when the insert is present the furcation plate is a bi-furcation plate or a tri-furcation plate to provide a uniform continuous phase flow within the annular region between the insert and the membrane. Most preferably the furcation plate is a tri-furcation plate.

The number of orifices provided in the insert may vary depending upon the injection rate, etc. Generally the number of orifices may be from 2 to 6. Preferably the number of orifice is three.

The chamfered region on the insert is advantageous in that it enables the insert to be centred when it is located in position inside the membrane. The external circumference of the ends of the insert has a minimal tolerance with the internal diameter of the tubular membrane. This enables the insert to be accurately centred, thereby providing a consistent annulus leading to a consistent laminar flow. Generally, the chamfered region will comprise a shallow chamfer, which is advantageous in that it evens the flow distribution and allows the use of orifices in the insert with larger cross-sectional area than could be achieved if the flow simply entered through orifices parallel to the axis of the insert. This keeps the fluid velocity down and therefore minimises unwanted pressure losses, and shear on the outlet. The distance between the start of the orifices and the start of the porous region on the tubular membrane allows an even velocity distribution to be established. The radial dimension of the insert is selected to provide an annular depth to provide a certain laminar flow for the flowrates chosen. The axial dimension is designed to generally give a combined orifice area which is greater than both the annular area and the inlet/exit tube area.

The use of membrane emulsification techniques in the preparation of lipid vesicles as herein described may comprise the use of turbulent flow, e.g. by stirring; or the use of laminar flow. In a particular aspect of the invention the membrane emulsification technique comprises the use of laminar flow, i.e. whilst generally avoiding or minimising any turbulent flow.

The use of membrane emulsification techniques in the preparation of lipid vesicles as herein described may include the use of one or more pump systems. It will be understood that any conventionally known pumping system for use with membrane emulsification may suitably be used. However, in a particular aspect of the invention the pump system may comprise a gear pump or a peristatic pump; and combinations thereof.

The lipid vesicles thus obtained have a high reproducibility both in the encapsulation rate and in the particle size distribution (polydispersity). The lipid vesicles may have a polydispersity index of ≤0.3, e.g. from about 0.05 to about 0.3.

The method of the invention can be used to precisely control the distribution of chemical conditions and mechanical forces so that they are constant on a length scale equivalent to that of a lipid vesicle. Hence, resultant lipid vesicle populations that are more uniform in size, hence of low polydispersity.

Polydispersity may be measured by the “Quasi Elastic Light Scattering” technique, which uses a laser radiation source in a photonic correlation spectrometer, provides the size distribution of the lipid vesicle population as well as the polydispersibility of the same, as measurement parameters of the homogeneity of the population.

In one aspect of the invention the crossflow apparatus includes an insert as herein described and the first inlet is a continuous phase first inlet and the second inlet is a disperse phase inlet; such that the disperse phase travels from outside the tubular membrane to inside.

In another aspect of the invention the crossflow apparatus does not include an insert and the first inlet is a disperse phase first inlet and the second inlet is a continuous phase inlet; such that the disperse phase travels from inside the tubular membrane to outside.

Separation, purification and/or dilution of the lipid vesicles might also be performed by any suitable method. Preferably the lipid vesicles are filtrated, more preferably the lipid vesicles are separated or purified by filtration through a sterile filter. For active loading and/or RNA loading dilution, in order to reduce the solvent concentration or to replace buffers may be followed by concentration by ultrafiltration.

Lipid vesicles, e.g. liposomes and lipid nanoparticles (LNPs), prepared by the method of the invention are useful as components in pharmaceutical compositions for immunising subjects against various diseases. These compositions will typically include a pharmaceutically acceptable carrier in addition to the lipid vesicle.

Therefore, according to a further aspect of the present invention there are provided lipid vesicles prepared by the method herein described. According to one aspect of the invention the lipid vesicles prepared by the method herein described are liposomes. According to another aspect of the invention the lipid vesicles prepared by the method herein described are LNPs.

According to this aspect of the invention the lipid vesicle may further include an active agent.

According to a yet further aspect of the present invention there is provided a composition. According to one aspect of the invention the composition comprises a liposome as herein described and a pharmaceutically acceptable excipient, carrier or diluent. According to another aspect of the invention the composition comprises an LNP as herein described and a pharmaceutically acceptable excipient, carrier or diluent.

As described herein the lipid vesicles prepared by the method of the invention described herein may suitably include a nucleic acid, such as one encoding for an antigen. Therefore, there is further provided a method of modulating the expression of a polypeptide by a cell, comprising providing to a cell a lipid vesicle, e.g. a liposome or a lipid nanoparticle (LNP), including a nucleic acid as herein described.

Thus according to a particular aspect of the invention the nucleic acid comprises a nucleic acid encoding an antigen. Therefore the present invention further provides a vaccine comprising a lipid vesicle and a nucleic acid encoding an antigen associated with a disease or pathogen as herein described.

By way of example only, active agents for use in the lipid vesicles of the present invention include, but shall not be limited to, biologically active agents, such as pharmaceutically active agents, vaccines and pesticides. Biologically active compounds may also include, for example, a plant nutritive substance or a plant growth regulant. Alternatively, the active agent may be non-biologically active, such as, a plant nutritive substance, a food flavouring, a fragrance, and the like.

Pharmaceutically active agents refer to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. Such active agents may be capable of preventing, alleviating, treating, and/or curing abnormal and/or pathological conditions of a living body, such as by destroying a parasitic organism, or by limiting the effect of a disease or abnormality by materially altering the physiology of the host or parasite. Such active agents may be capable of maintaining, increasing, decreasing, limiting, or destroying a physiologic body function. Active agents may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. Active agents may be capable of otherwise treating (such as deodorising, protecting, adorning, grooming) a body. Depending upon the effect and/or its application, the active agent may further be referred to as a bioactive agent, a pharmaceutical agent (such as a prophylactic agent, or a therapeutic agent), a diagnostic agent, a nutritional supplement, and/or a cosmetic agent, and includes, without limitation, prodrugs, affinity molecules, synthetic organic molecules, polymers, molecules with a molecular weight of 2 kD or less (such as 1.5 kD or less, or 1 kD or less), macromolecules (such as those having a molecular weight of 2 kD or greater, preferably 5 kD or greater), proteinaceous compounds, peptides, vitamins, steroids, steroid analogues, nucleic acids, carbohydrates, precursors thereof and derivatives thereof. Active agents may be ionic, non-ionic, neutral, positively charged, negatively charged, or zwitterionic, and may be used singly or in combination of two or more thereof. Active agents may be water insoluble or water soluble.

The term “macromolecule” used herein refers to a material capable of providing a three-dimensional (e.g., tertiary and/or quaternary) structure.

A wide variety of pharmaceutically active agents may be utilised in the present invention. Thus, the pharmaceutically active agent may comprise one or more of a polynucleotide, a peptide, a protein, a small organic active agent, a small inorganic active agent and mixtures thereof.

A polynucleotide active agent may comprise one or more of an oligonucleotide, an antisense construct, a siRNA, an enzymatic RNA, a recombinant DNA construct, an expression vector, and mixtures thereof. The lipid vesicle delivery system of the present invention may be useful for in vivo or in vitro delivery of active agents, such as, amino acids, peptides and proteins. Peptides can be signalling molecules such as hormones, neurotransmitters or neuromodulators, and can be the active fragments of larger molecules, such as receptors, enzymes or nucleic acid binding proteins. The proteins can be enzymes, structural proteins, signalling proteins or nucleic acid binding proteins, such as transcription factors.

When the pharmaceutically active agent comprises a small organic active agent it may comprise a therapeutic agent or a diagnostic agent. In particular embodiments a small organic active agent may comprise a sequence-specific DNA binding oligomer, an oligomer of heterocyclic polyamides, for example, those disclosed in U.S. Pat. No. 6,506,906 which is hereby incorporated by reference. Other small organic active agents may comprise those disclosed by Dervan in “Molecular Recognition of DNA by Small Molecules, Bioorganic & Medicinal Chemistry (2001) 9: 2215-2235”, which is hereby incorporated by reference. In certain embodiments, the oligomer may comprise monomeric subunits selected from the group consisting of N-methylimidazole carboxamide, N-methylpyrrole carboxamide, beta-alanine and dimethyl aminopropylamide.

In another embodiment of the present invention the lipid vesicle delivery system of the present invention may include an inorganic active agent, e.g. gastrointestinal therapeutic agents such as aluminium hydroxide, calcium carbonate, magnesium carbonate, sodium carbonate and the like.

In another embodiment of the invention, more than one type of polynucleotide may be enclosed within the lipid vesicle delivery system. Such polynucleotides provide the ability to express multiple gene products under control, in certain embodiments, at least one expressible gene product is a membrane protein, such as a membrane receptor, most preferably a membrane-bound receptor for a signalling molecule. In some embodiments, at least one expressible gene product is a soluble protein, such as a secreted protein, e.g. a signalling protein or peptide.

The present invention also provides a method of immunising an individual against a pathogen. The method may comprise the step of contacting cells of said individual with a lipid vesicle, e.g. a lipid nanoparticle, delivery system comprising a lipid vesicle and a nucleic acid composition, thereby administering to the cells a nucleic acid molecule that comprises a nucleotide sequence that encodes a peptide which comprises at least an epitope identical to, or substantially similar to, an epitope displayed on said pathogen as antigen, and said nucleotide sequence is operatively linked to regulatory sequences, wherein the nucleic acid molecule is capable of being expressed in the cells of the individual. In another embodiment, the present invention provides a method of producing immunity to a toxoid comprising the steps of providing a lipid vesicle delivery system comprising a lipid vesicle and a toxoid, contacting a phagocytic cell with the lipid vesicle delivery system and inducing phagocytosis of the lipid vesicle delivery system. The phagocytic cell can be one or more of macrophages, M cells of the Peyer's patches, monocytes, neutrophils, dendritic cells, Langerhans cells, Kupffer cells, alveolar phagocytes, peritoneal macrophages, milk macrophages, microglia, eosinophils, granulocytes, mesengial phagocytes, and synovial A cells.

Lipid vesicle compositions according to this aspect of the invention may be suitable for the delivery of active agents in a variety of clinical areas including, but not limited to, anti-cancer, anti-fungal and anti-inflammatory therapies; and therapeutic genes. Clinically available lipid vesicle formulations include doxorubicin (Doxil®), amphotericin (Ambisome®) and extended release morphine (DepoDur™) can be prepared according to the methods described herein.

The present invention will now be described by way of example only, with reference to the accompanying Examples and Figures in which:

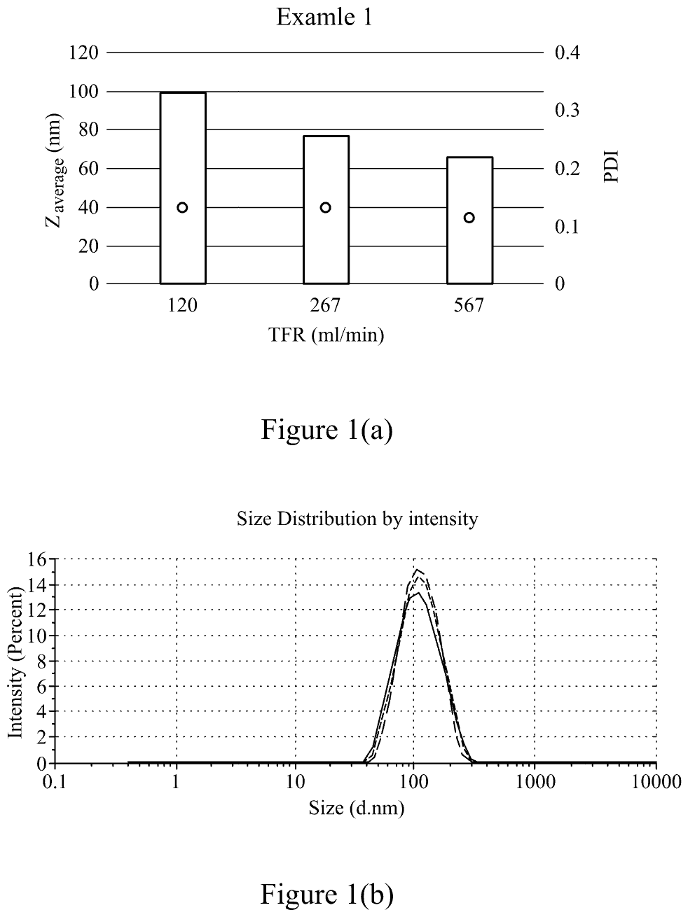

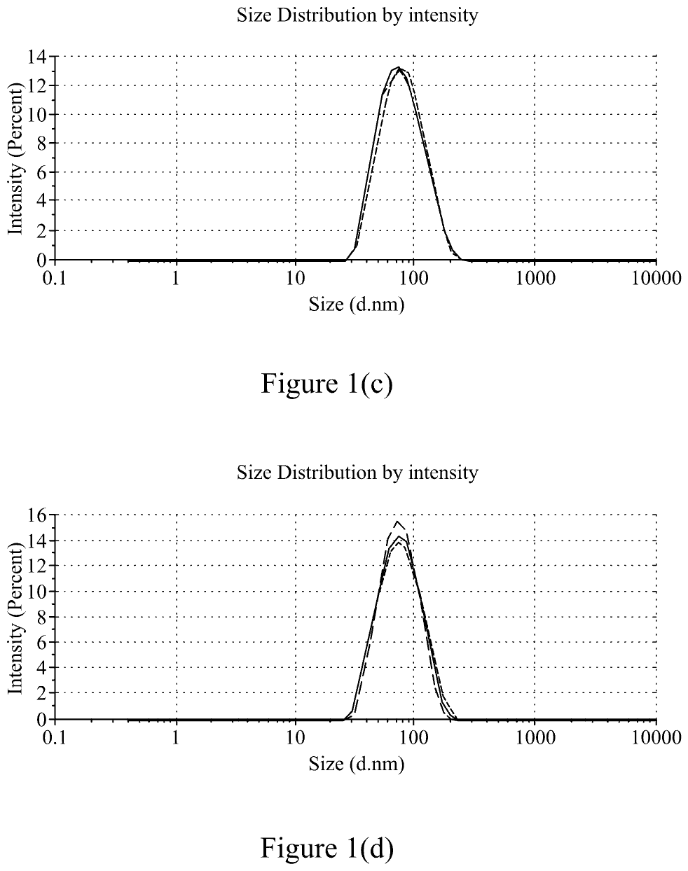

FIG. 1(a) illustrates the effect of Total Flow Rate (TFR) on particle size for unloaded liposomes;

FIGS. 1(b)-(d) illustrate the size distribution by intensity for unloaded liposomes at flow rates of 120 mL/min, 267 mL/min and 567 mL/min, respectively;

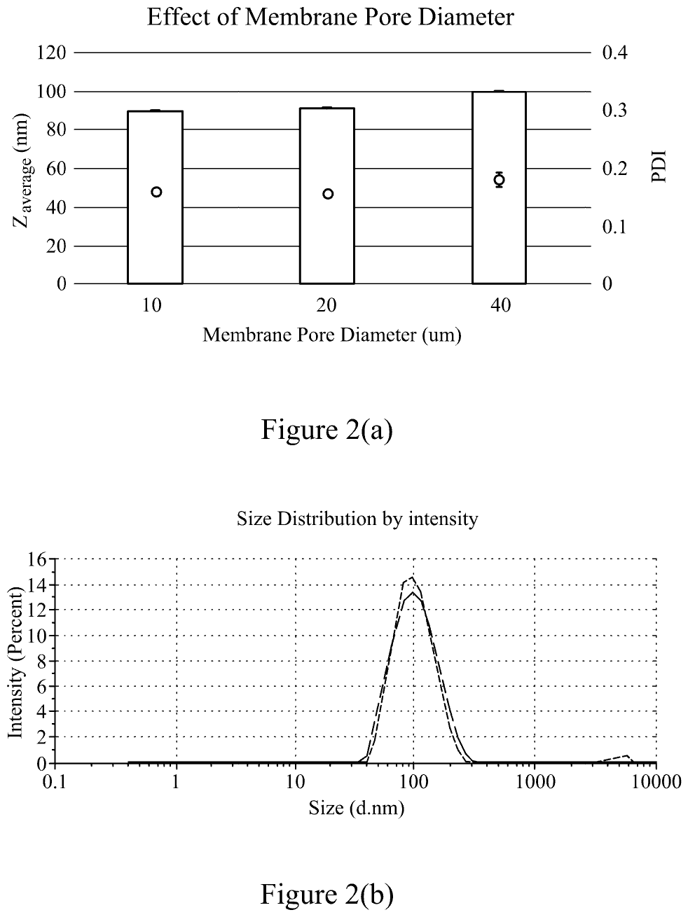

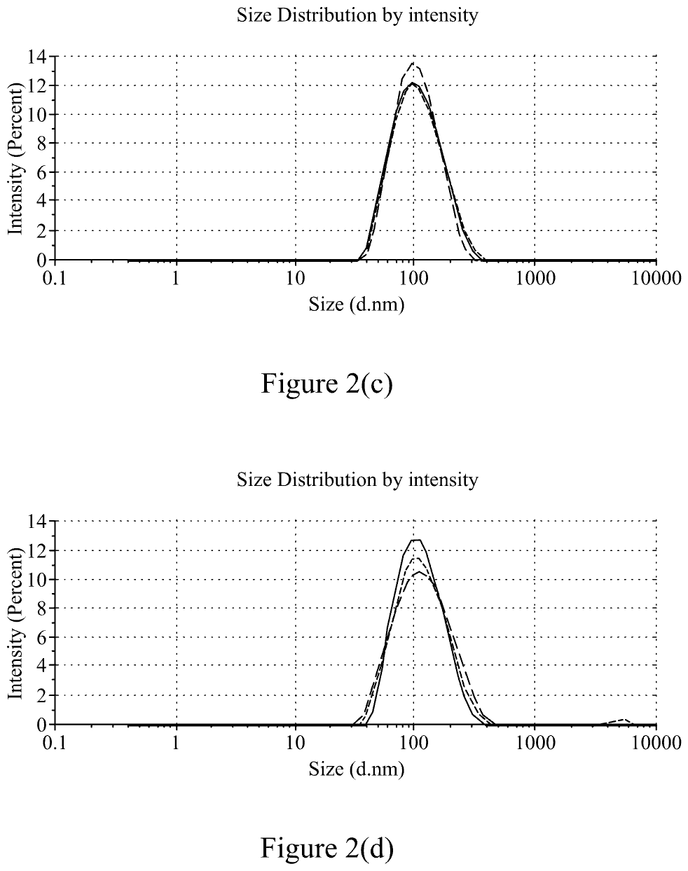

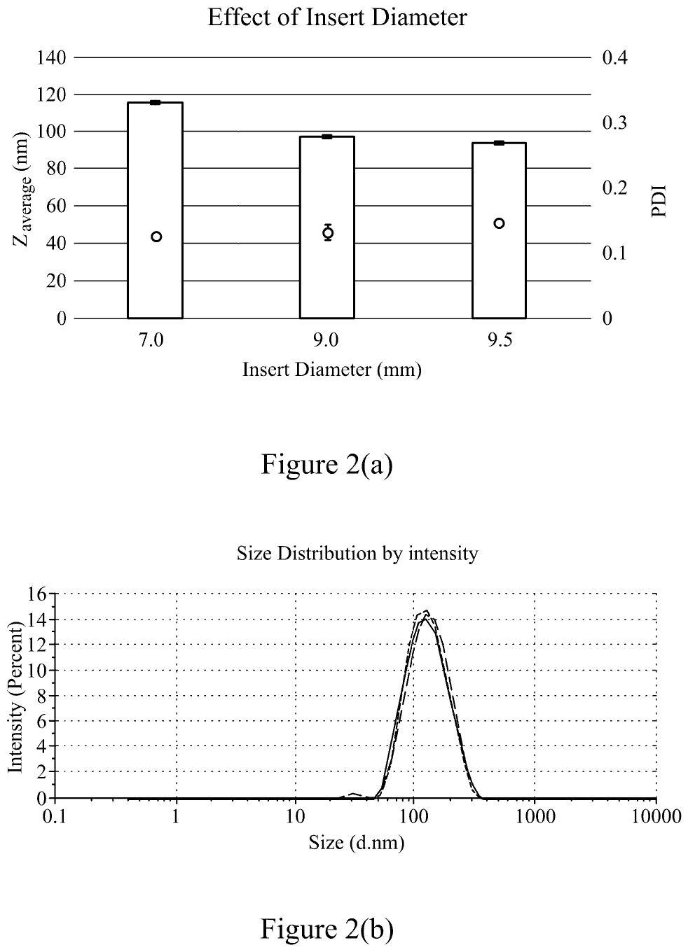

FIG. 2(a) illustrates the effect of Membrane Pore Diameter on particle size for unloaded liposomes;

FIGS. 2(b)-(d) illustrate the size distribution by intensity for unloaded liposomes at membrane pore diameters of 10 μm, 20 μm and 40 μm respectively;

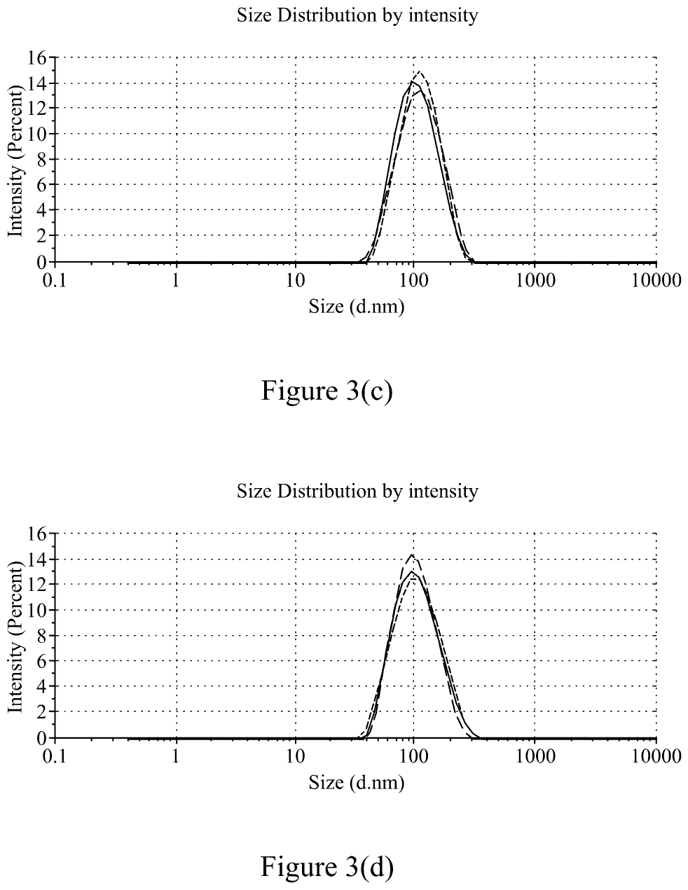

FIGS. 3(a)-(d) illustrate the size distribution by intensity for unloaded liposomes at insert diameters of 7 mm, 9 mm and 9.5 mm respectively;

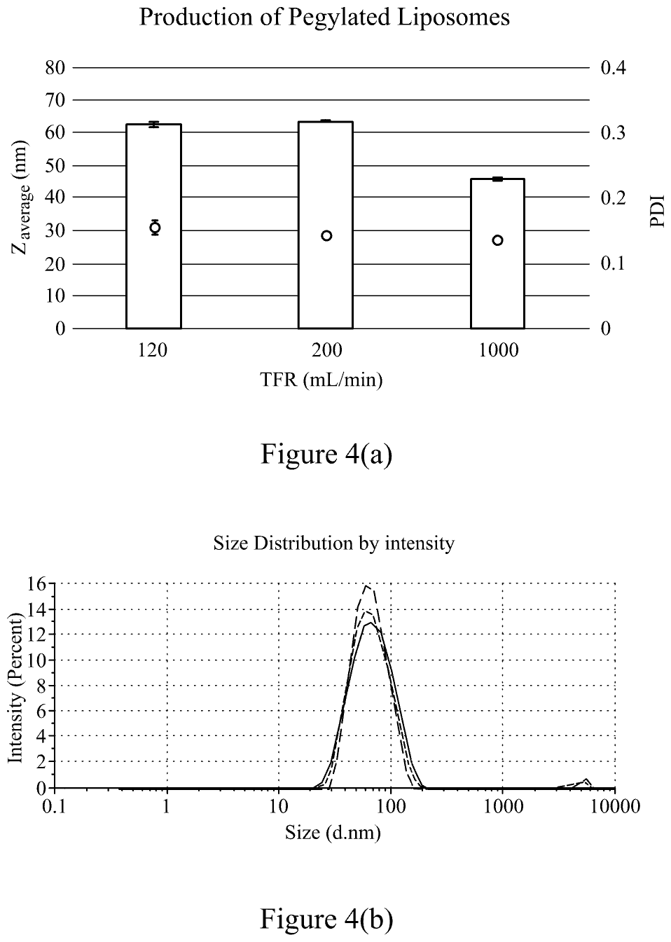

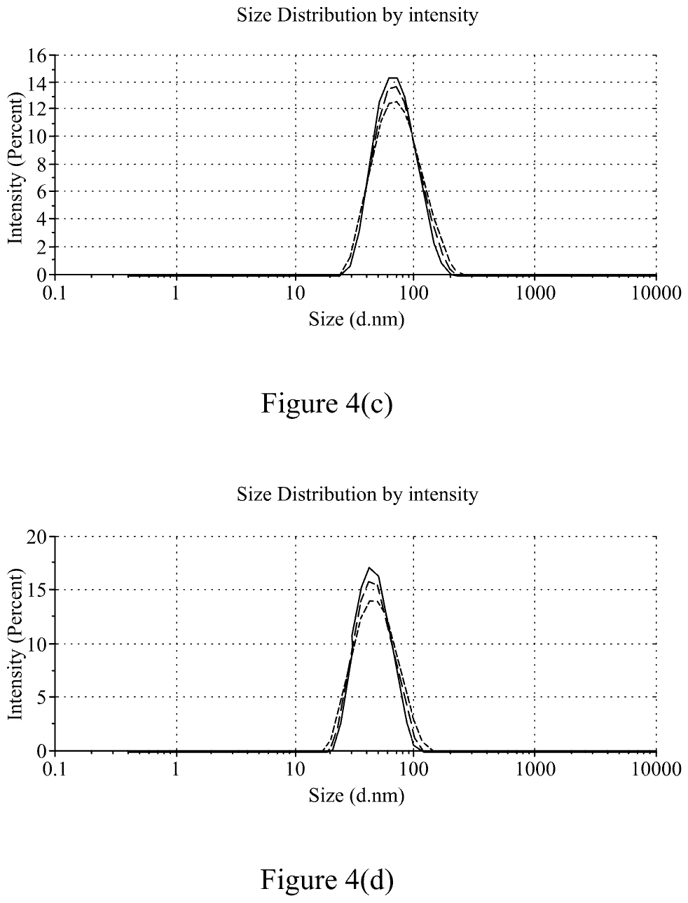

FIG. 4(a) illustrates the effect of Total Flow Rate (TFR) on particle size for pegylated liposomes;

FIGS. 4(b)-(d) illustrate the size distribution by intensity for pegylated liposomes at flow rates of 120 mL/min, 200 mL/min and 1,000 mL/min, respectively;

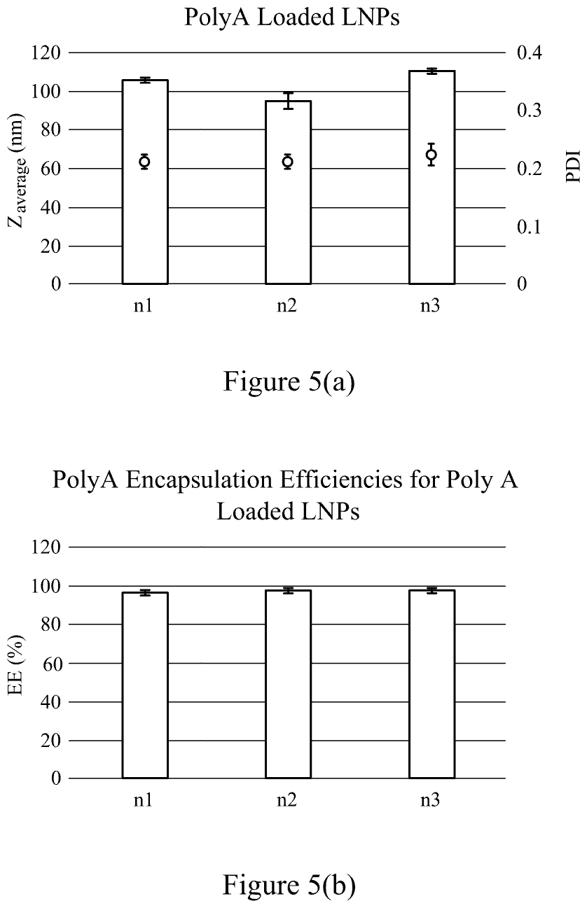

FIGS. 5(a) and (b) illustrate the particle size verses the polydispersity index (PDI) for LNPs loaded with an RNA analogue flow rate on particle size verses the polydispersity index (PDI) for LNPs loaded with an RNA analogue at a lipid phase flow rate of 125 mL/min; and

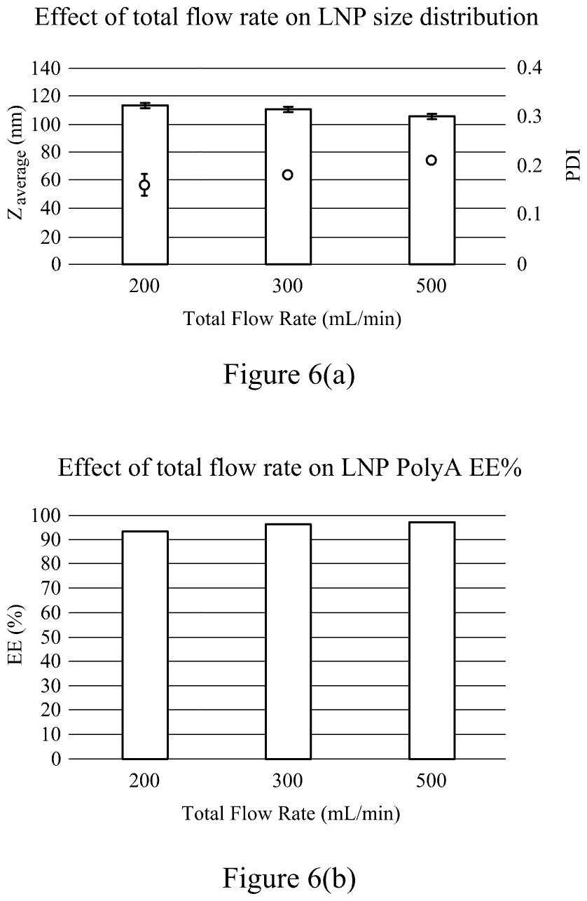

FIGS. 6(a) and (b) illustrate the effect of flow rate on particle size verses the polydispersity index (PDI) for LNPs loaded with an RNA analogue at flow rates of 200 mL/min, 300 mL/min and 500 mL/min respectively.

### EXAMPLES

### Example 1

Effect of Flow Rate on Particle Size of Unloaded Liposomes

A vessel containing an aqueous PBS buffer was prepared, alongside another vessel containing a solution of lipids and cholesterol in ethanol at a total concentration of 20 mg/mL.

The AXF micromixing equipment consisted of the device housing, a membrane with 10 μm pores and a spacing of 200 μm in a square grid, and an insert 9.5 mm in diameter.

Peristaltic pumps were used to pump both phases, using tygon tubing. The aqueous buffer phase was pumped through the centre of the AXF membrane micromixing equipment, at rates of 96, 214 and 454 mL/min. The lipid phase was pumped at rates of 24, 53 and 113 mL/min, into the top port of the device, through the membrane, and into the aqueous phase flow, maintaining the 4:1 aqueous:organic phase ratios across all the experiments.

The resultant 20% ethanol solution was further diluted by the addition of aqueous PBS buffer, and the resulting dilute solution was concentrated via ultrafiltration.

The resulting suspension was analysed via Dynamic Light Scattering/Quasi-Elastic Light Scattering (DLS/QELS), and intensity Z_{average} particle size and PDI values were recorded and reported in Table 1 and FIGS. 1(a)-(d).

TABLE 1

| Total flow rate (TFR) | Z average (nm) | PDI |
|---|---|---|
| 120 | 99.35 | 0.13 |
| 267 | 76.37 | 0.134 |
| 567 | 65.32 | 0.115 |

### Example 2

Effect of Membrane Pore Size on Particle Size and Distribution of Unloaded Liposomes

A vessel containing an aqueous PBS buffer was prepared, alongside another vessel containing a solution of lipids and cholesterol in ethanol at a total concentration of 20 mg/mL.

The AXF micromixing equipment consisted of the device housing, a membrane, and an insert 9.5 mm in diameter. 3 membranes were used, with pore diameters of 10, 20 and 40 μm. All had a pore spacing (pitch) of 200 μm in a square grid.

Peristaltic pumps were used to pump both phases, using tygon tubing. The aqueous buffer phase was pumped through the centre of the AXF membrane micromixing equipment, at a rate of 240 mL/min. The lipid phase was pumped at rates of 40 mL/min, into the top port of the device, through the membrane, and into the aqueous phase flow, giving a 6:1 aqueous:organic phase ratio.

The resultant ˜14% ethanol solution was further diluted by the addition of aqueous PBS buffer, and the resulting dilute solution was concentrated via ultrafiltration.

The resulting suspension was analysed via DLS/QELS, and intensity Z_{average} particle size and PDI values were recorded and reported in Table 2 and FIGS. 2(a)-(d).

TABLE 2

| Membrane Pore Size (μm) | Z average (nm) | PDI |
|---|---|---|
| 10 | 89.68 ± 0.44 | 0.156 ± 0.006 |
| 20 | 91.24 ± 0.14 | 0.153 ± 0.006 |
| 40 | 99.63 ± 0.24 | 0.178 ± 0.013 |

### Example 3

Effect of Insert Diameter on Particle Size and Distribution of Unloaded Liposomes A vessel containing an aqueous PBS buffer was prepared, alongside another vessel containing a solution of lipids and cholesterol in ethanol at a total concentration of 20 mg/mL.

The AXF micromixing equipment consisted of the device housing, a membrane with a pore diameter of 10 μm and a pore spacing (pitch) of 200 μm in a square grid, and an insert. 3 inserts were used, with diameters of 7.0 mm, 9.0 mm and 9.5 mm.

Peristaltic pumps were used to pump both phases, using tygon tubing. The aqueous buffer phase was pumped through the centre of the AXF membrane micromixing equipment, at a rate of 240 mL/min. The lipid phase was pumped at rates of 40 mL/min, into the top port of the device, through the membrane, and into the aqueous phase flow, giving a 6:1 aqueous:organic phase ratio.

The resultant ˜14% ethanol solution was further diluted by the addition of aqueous PBS buffer, and the resulting dilute solution was concentrated via ultrafiltration.

The resulting suspension was analysed via DLS/QELS, and intensity Z_{average} particle size and PDI values were recorded and reported in Table 3 and FIGS. 3(a)-(d).

TABLE 3

| Insert Diameter (mm) | Z average (nm) | PDI |
|---|---|---|
| 7.0 | 114.10 ± 0.85 | 0.121 ± 0.003 |
| 9.0 | 95.97 ± 1.13 | 0.142 ± 0.011 |
| 9.5 | 92.92 ± 0.30 | 0.144 ± 0.007 |

### Example 4

Production of Pegylated Liposomes

A vessel containing an aqueous HEPEs buffer (10 mM, pH 7.4) was prepared, alongside another vessel containing a solution of lipids and cholesterol in ethanol. The lipids were HSPC (S PC-3, Lipoid GmBH), DSPC-mPEG2000 (Lipoid GmBH), and cholesterol (Sigma Aldrich) at a molar ratio of HSPC/Cholesterol/Pegylated Lipid of 56.2/38.5/5.3, at a total concentration of 10 mg/mL.

The AXF micromixing equipment consisted of the device housing, a membrane with 10 μm pores and a spacing of 200 μm in a square grid, and a 9.5 mm insert.

Peristaltic pumps were used to pump both phases, using tygon tubing. Both phases were held above the T_{c} of the lipids. The aqueous buffer phase was pumped through the centre of the AXF membrane micromixing equipment, at rates of 90 mL/min, 150 mL/min and 750 mL/min. The lipid phase was pumped at rates of 30 mL/min, 50 mL/min and 250 mL/min, into the top port of the device, through the membrane, and into the aqueous phase flow, giving a 3:1 aqueous:organic phase ratio.

The resultant 25% ethanol solution was further diluted by the immediate addition of aqueous HEPEs buffer, and the resulting dilute solution was concentrated via ultrafiltration.

The resulting suspension was analysed via DLS/QELS, and intensity Z_{average} particle size and PDI values were recorded and reported in Table 3 and FIGS. 4(a)-(d).

TABLE 3

| Total Flow Rate (mL/min) | Z average (nm) | PDI |
|---|---|---|
| 120 | 62.40 ± 0.76 | 0.153 ± 0.014 |
| 200 | 63.30 ± 0.18 | 0.142 ± 0.008 |
| 1000 | 45.64 ± 0.33 | 0.132 ± 0.003 |

### Example 5

Reproducibility in Production of LNPs Loaded with an RNA Analogue

A vessel containing an aqueous 100 mM citrate buffer system (pH 6) and the RNA analogue polyA was prepared, alongside another vessel containing a solution of lipids in ethanol. The lipids were the cationic lipid DDAB, the structural lipid DSPC, the pegylated lipid DMG-PEG2000 and cholesterol, at a total lipid concentration of 3 mM and a molar ratio of DDAB/DSPC/Chol/DMG-PEG2000 of 40/10/48/2. The nitrogen-to-phosphate ratio (N/P; nitrogen from the cationic lipid and phosphate from the nucleic acid) was 6.

The AXF micromixing equipment consisted of the device housing, a membrane with 10 μm pores and a spacing of 200 μm in a square grid, and an insert 9.0 mm in diameter.

Gear pumps were used to pump both phases, using PFA tubing. The aqueous buffer phase was pumped through the centre of the AXF membrane micromixing equipment, at a rate of 375 mL/min. The lipid phase was pumped at a rate of 125 mL/min, into the top port of the device, through the membrane, and into the aqueous phase flow, giving a 3:1 aqueous:organic phase ratio.

The resultant 25% ethanol solution was further diluted by the addition of aqueous buffer, and the resulting dilute solution was concentrated via ultrafiltration.

The experiment was run 3 times, and the resulting suspensions were analysed via DLS/QELS, and intensity Z_{average} particle size and PDI values were recorded. Nucleic acid loading was quantified by Ribogreen assay. These values are reported in Table 4 and FIGS. 5(a)-(b).

TABLE 4

| Run | Z average (nm) | PDI | EE (%) |
|---|---|---|---|
| n1 | 105.03 ± 1.01 | 0.21 ± 0.010 | 96.45 ± 0.33 |
| n2 | 94.01 ± 3.83 | 0.21 ± 0.010 | 97.81 ± 0.29 |
| n3 | 109.57 ± 0.25 | 0.22 ± 0.018 | 97.09 ± 0.39 |

### Example 6

Effect of Flow Rate in the Production of LNPs Loaded with an RNA Analogue

A vessel containing an aqueous 100 mM citrate buffer system (pH 6) and the RNA analogue polyA was prepared, alongside another vessel containing a solution of lipids in ethanol. The lipids were the cationic lipid DDAB, the structural lipid DSPC, the pegylated lipid DMG-PEG2000 and cholesterol, at a total lipid concentration of 3 mM and a molar ratio of DDAB/DSPC/Chol/DMG-PEG2000 of 40/10/48/2. The nitrogen-to-phosphate ratio (N/P; nitrogen from the cationic lipid and phosphate from the nucleic acid) was 6.

The AXF micromixing equipment consisted of the device housing, a membrane with 10 μm pores and a spacing of 200 μm in a square grid, and an insert 9.0 mm in diameter.

Gear pumps were used to pump both phases, using PFA tubing. The aqueous buffer phase was pumped through the centre of the AXF membrane micromixing equipment. The lipid phase was pumped into the top port of the device, through the membrane, and into the aqueous phase flow. The total flow rates were 100 mL/min, 200 mL/min, 300 mL/min and 500 mL/min. A 3:1 aqueous:organic phase ratio was maintained for all runs.

The resultant 25% ethanol solution was further diluted by the addition of aqueous buffer, and the resulting dilute solution was concentrated via ultrafiltration.

The experiment was run 3 times, and the resulting suspensions were analysed via DLS/QELS, and intensity Z_{average} particle size and PDI values were recorded. Nucleic acid loading and encapsulation efficiency (EE) was quantified by Ribogreen assay. All values are reported in Table 5 and FIGS. 6(a) and (b).

TABLE 5

| Total Flow Rate (mL/min) | Z average (nm) | PDI | EE (%) |
|---|---|---|---|
| 200 | 113.7 ± 1.65 | 0.160 ± 0.019 | 93.80 |
| 300 | 110.55 ± 1.62 | 0.183 ± 0.002 | 97.10 |
| 500 | 105.23 ± 1.00 | 0.214 ± 0.008 | 97.45 |

1. A method of preparing lipid vesicles, said method comprising dispersing a first liquid phase in a second liquid phase;
wherein said first liquid phase comprises a lipid phase and said second liquid phase comprises an aqueous phase; or said first liquid phase comprises an aqueous phase and said second liquid phase comprises a lipid phase;
said method comprising controlling provision of the first liquid phase in a first flow direction to a membrane, said membrane defining a plurality of pores; and controlling provision of the second liquid phase to the membrane in a crossflow to the first flow direction, via the plurality of pores, to form a lipid vesicle suspension.

2. The method according to claim 1 wherein the first liquid phase comprises a lipid phase and the second liquid phase comprises an aqueous phase.

3. The method according to claim 1 wherein the first liquid phase comprises an aqueous phase and the second liquid phase comprises a lipid phase.

4. The method according to claim 1 wherein the lipid vesicles are liposomes or lipid nanoparticles (LNPs).

5. (canceled)

6. (canceled)

7. A method of preparing lipid vesicles, said method comprising dispersing a first liquid phase in a second liquid phase, wherein said first liquid phase comprises a lipid phase;
wherein said method uses a crossflow emulsification apparatus; said crossflow emulsification apparatus (AXF) comprising:
an outer tubular sleeve provided with a first inlet at a first end; a lipid vesicle suspension outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate; and
controlling provision of the first liquid phase to the tubular membrane; and controlling provision of a second liquid phase to the tubular membrane via the plurality of pores to form a lipid vesicle suspension.

8. (canceled)

9. (canceled)

10. The method according to claim 7 wherein the lipid vesicles are liposomes or lipid nanoparticles (LNPs).

11. (canceled)

12. (canceled)

13. The method according to claim 1 wherein the aqueous phase includes one or more active agents.

14. The method according to claim 13 wherein the aqueous phase comprises a buffered solution.

15. The method according to claim 14 wherein the aqueous phase buffers include, but shall not be limited to, MES (2-N-morpholino)ethanesulfonic acid), citrate, phosphate, acetate, HEPES (4-(2-hydroxyethyl)-1-piperazineethanesulfonic acid), TRIS (tris(hydroxymethyl)aminomethane) and PBS (phosphate-buffered saline); and combinations thereof.

16. The method according to claim 7 wherein when the one or more active agents is hydrophobic, then the one or more active agents may be included in the lipid phase.

17. (canceled)

18. The method according to claim 1 wherein the lipid vesicles are produced unloaded and loaded afterwards (active loading).

19. The method according to claim 1 wherein the lipid vesicles are produced loaded (passive loading).

20. (canceled)

21. The method according to claim 13 wherein the one or more active agents is a bioactive agents, such as a therapeutic agent (drug), vaccine and the like.

22. The method according to claim 21 wherein the bioactive agent is a therapeutic nucleic acid such as one encoding for an antigen.

23. The method according to claim 22 wherein therapeutic nucleic acids include, e.g., messenger RNA (mRNA), antisense oligonucleotides, ribozymes, DNAzymes, plasmids, immune stimulating nucleic acids, antagomir, antimir, mimic, supermir and aptamers.

24. (canceled)

25. (canceled)

26. The method according to claim 1 wherein the lipid vesicles are LNPs and the LNPs are ionisable or cationic LNPs.

27. The method according to claim 1 wherein the lipid vesicles comprise cationic lipid vesicles, such as, DDA (dimethyl dioctadecyl ammonium bromide) or DOTAP (1,2-dioleoyl-3-trimethylammonium-propane) may suitably be used.

28. (canceled)

29. The method according to claim 1 wherein the lipid vesicles comprise neutral lipid vesicles and the neutral lipid vesicles comprise sphingosylphosphorylcholine (SPC), L-α-hydrogenated phosphatidylcholine (HSPC), distearoylphosphatidylcholine (DSPC) and 1-palmitoyl-2-oleoylphosphatidylcholine, (POPC), and the like; and combinations thereof.

30. (canceled)

31. The method according to claim 1 wherein the lipid vesicles comprise a lipid having a pKa in the range of 5.0 to 7.6 and the lipids includes a tertiary amine.

32. The method according to claim 31 wherein the lipid vesicles comprise 1,2-dilinoleyloxy-N,N-dimethyl-3-aminopropane or 1,2-dilinolenyloxy-N,N-dimethyl-3-aminopropane. Another suitable lipid having a tertiary amine is 1,2-dioleyloxy-N,Ndimethyl-3-aminopropane.

33.-35. (canceled)

36. The method according to claim 1 wherein the lipid vesicles comprise a pegylated lipid.

37. The method according to claim 36 wherein the pegylated lipids include, but shall not be limited to, 1-(monomethoxy-polyethyleneglycol)-2,3-dimyristoylglycerol (PEG-DMG), pegylated diacylglycerol (PEG-DAG), e.g. 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG), e.g. 4-O-(2′,3′-di(tetradecanoyloxy)propyl-1-O-(\acute{ω}-methoxy(polyethoxy)ethyl)butanedioate (PEG-S-DMG), a pegylated ceramide (PEG-cer), or a PEG dialkoxypropylcarbamate, e.g. \acute{ω}-methoxy(polyethoxy)ethyl-N-(2,3-di(tetradecanoxy)propyl)carbamate, 2,3-di(tetradecanoxy)propyl-N-(\acute{ω}-methoxy(polyethoxy)ethyl)carbamate or distearoyl-sn-glycero-3-phosphoethanolamine-N-[amino(polyethylene glycol) 2000]; and combinations thereof.

38. (canceled)

39. (canceled)

40. A method according to claim 7 wherein the apparatus includes an insert.

41.-51. (canceled)

52. A method according to claim 7 wherein the crossflow apparatus comprises a plurality of tubular membranes.

53.-79. (canceled)

80. A method according to claim 7 wherein the apparatus is suitable for preparing lipid vesicles with a PDI of from about 0.02 to about 0.3.

81. Lipid vesicles prepared by the method according to claim 1.

82.-99. (canceled)
